(19) United States
(12) Patent Application Publication
(10) Pub. No.: US 2019/0392923 A1
(43) Pub. Date: Dec. 26, 2019

(54) System for Annotating a Patient Record

(71) Applicant: Gary P. Warren, Williamsburg, VA (US)

(72) Inventor: Gary P. Warren, Williamsburg, VA (US)

(21) Appl. No.: 15/998,055

(22) Filed: Jun. 21, 2018

(51) Int. Cl. G16H 10/60; A61M 5/168

(52) U.S. Cl. CPC G16H 10/60; A61M 2205/502; A61M 5/16836

(57) ABSTRACT

An annotation system for an electronic medical record of a patient, including a user interface controlling a patient monitoring device. The patient monitoring device aids detection of infiltration or extravasation at the patient's cannulation site. The user interface includes a check button registering periodic examinations of the cannulation site. The first key annotates the electronic medical record with an indication that the cannulation site is healthy. The second key annotates the electronic medical record with an indication that infiltration or extravasation is indicated at the cannulation site.

### CROSS-REFERENCES TO RELATED APPLICATIONS

Not Applicable.

### STATEMENT REGARDING FEDERALLY SPONSORED RESEARCH OR DEVELOPMENT

Not Applicable

### BACKGROUND OF THE INVENTION

A basic tenet in the field of medicine is giving care. In many situations, it may be desirable to have one or more caregivers continuously evaluate a patient, i.e., provide “round the clock care.” However, such continuous care is frequently impractical because it is cost prohibitive or qualified caregivers are not available. Typically, evaluation of a patient is intermittently performed on an “as-available” basis of the caregiver.

Caregiver evaluations often involve quantitative measurement of vital signs. Pulse rate, blood oxygenation, blood pressure, and body temperature are examples of vital signs. Caregivers use medical devices such as pulse oximeters, sphygmomanometers, and thermometers for measuring a patient's vital signs. These devices provide caregivers with numerical values that characterize the patient's vital signs, and these values may be compared to known ranges to evaluate the health of a patient.

Caregiver evaluations may also involve qualitative judgements about the patient. Examples of common qualitative judgements for evaluating a patient may include interviews, observations such as direct comparisons, and tactile examinations.

As the phrase is used herein, a medical record (also known as a health record or medical chart) describes the body of information obtained from systematically documenting a single patient's medical history and care over time. In addition to qualitative evaluations, the medical record includes a variety of qualitative evaluations that are entered by professional caregivers. These notes, for example, record observations, administration of drugs and therapies, orders for the administration of drugs and therapies, test results, x-rays, etc. The maintenance of complete and accurate medical records is a requirement of health care providers and is generally a prerequisite for licensing or certification of professional caregivers. When referring to medical records, information about an individual patient may be stored in physical folders and/or electronic records.

Typically, a professional caregiver, e.g., nurse, is concurrently responsible for numerous patients. Accurately maintaining each patient's medical record requires attention to detail and can be quite burdensome for the nurse. It may be that a nurse needs only to annotate the patient's medical record with an indication that the patient is “okay.” Such an indication may be a generalization of a more explicit indication of the patient's status, such as “patient appears to be progressing as anticipated” or “patient is showing no adverse symptoms.” At other times, a nurse may wish to set a place-holder in the patient's medical record to subsequently detail an unusual or adverse development or when an additional action is to be taken. The nurse's place-holder may be a general indication such as “not okay,” which the nurse may subsequently revise with a more detailed explanation of their evaluation, treatment or recommendation.”

If annotating each individual patient's medical record takes just two minutes per hour for a nurse that is assigned eight patients, then more that 25% of the nurse's time is spent documenting patients' care. This means that, on average, the nurse can provide less than six minutes of actual care to each patient per hour. If one patient requires 15 minutes of attention in one hour, then two of the nurse's eight patients will not be afforded their care that hour.

### BRIEF SUMMARY OF THE INVENTION

Embodiments according to the present invention include an annotation system for an electronic medical record of a patient. The system includes a patient monitoring device and a user interface controlling the patient monitoring device. The patient monitoring device aids detection of at least one of infiltration and extravasation at a cannulation site of the patient. The user interface includes a check button, a first key, and a second key. The check button has at least one regularly assigned function including registering periodic examinations of the cannulation site. The first key annotates the electronic medical record with an indication that the cannulation site is healthy. The second key annotates the electronic medical record with an indication that at least one of infiltration and extravasation is indicated at a cannulation site.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features, principles, and methods of the invention.

FIG. 1A is a schematic view illustrating a typical set-up for infusion administration.

FIG. 1B is a schematic view illustrating a subcutaneous detail of the set-up shown in FIG. 1A.

FIGS. 2A-2C are perspective views illustrating the physical features of a known patient monitoring device.

FIG. 3 is a schematic view illustrating an embodiment according to the present disclosure of a user interface for a medical record annotation system.

FIG. 4 is a schematic view of the user interface for a medical record annotation system shown in FIG. 3.

FIG. 5 is a schematic view of the user interface for a medical record annotation system shown in FIG. 4.

FIG. 6 is a schematic view of the user interface for a medical record annotation system shown in FIG. 3.

FIG. 7 is a schematic view of the user interface for a medical record annotation system shown in FIG. 6.

FIG. 8 is a schematic view illustrating another embodiment according to the present disclosure of a user interface for a medical record annotation system.

FIG. 9 is a schematic view of the user interface for a medical record annotation system shown in FIG. 8.

FIG. 10 is a schematic view of the user interface for a medical record annotation system shown in FIG. 9.

In the figures, the thickness and configuration of components may be exaggerated for clarity. The same reference numerals in different figures represent the same component.

### DETAILED DESCRIPTION OF THE INVENTION

The following description and drawings are illustrative and are not to be construed as limiting. Numerous specific details are described to provide a thorough understanding of the disclosure. However, in certain instances, well-known or conventional details are not described in order to avoid obscuring the description.

Reference in this specification to “one embodiment” or “an embodiment” means that a particular feature, structure, or characteristic described in connection with the embodiment is included in at least one embodiment according to the disclosure. The appearances of the phrases “one embodiment” or “other embodiments” in various places in the specification are not necessarily all referring to the same embodiment, nor are separate or alternative embodiments mutually exclusive of other embodiments. Moreover, various features are described that may be exhibited by some embodiments and not by others. Similarly, various features are described that may be included in some embodiments but not other embodiments.

The terms used in this specification generally have their ordinary meanings in the art, within the context of the disclosure, and in the specific context where each term is used. Certain terms in this specification may be used to provide additional guidance regarding the description of the disclosure. It will be appreciated that a feature may be described more than one-way.

Alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term.

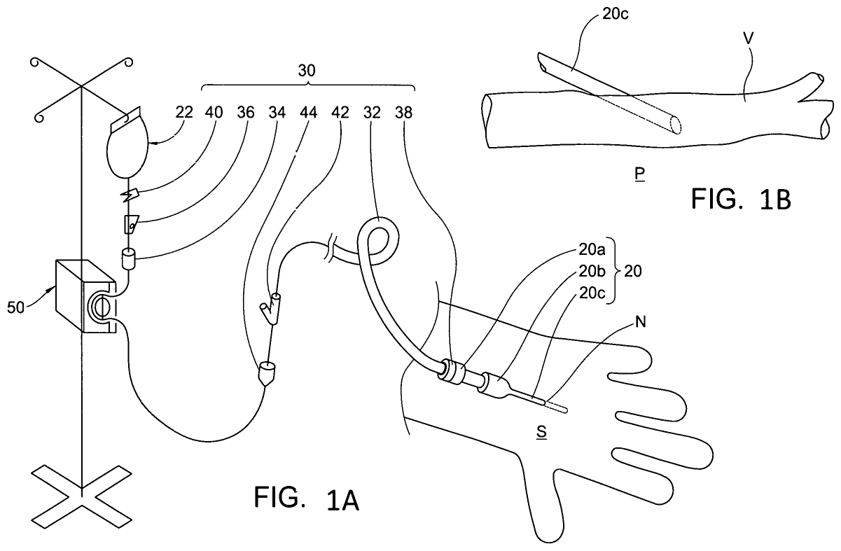

One of the most common procedures administered to patients by professional caregivers is an intravascular infusion. FIGS. 1A and 1B show a typical arrangement for intravascular infusion. As the terminology is used herein, “intravascular” preferably refers to being situated in, occurring in, or being administered by entry into a blood vessel, thus “intravascular infusion” preferably refers to introducing a fluid or infusate into a subcutaneous blood vessel V. Intravascular infusion accordingly encompasses both intravenous infusion (administering a fluid into a vein) and intra-arterial infusion (administering a fluid into an artery).

A cannula 20 typically is used for administering fluid via the blood vessel V. Typically, cannula 20 is inserted through skin S at a cannulation site N and punctures the blood vessel V, for example, the cephalic vein, basilica vein, median cubital vein, or any suitable vein for an intravenous infusion. Similarly, any suitable artery may be used for an intra-arterial infusion.

Cannula 20 typically is in fluid communication with a fluid source 22. Typically, cannula 20 includes an extracorporeal connector 20a, a hub 20b, and a transcutaneous sleeve 20c. An extension tube may couple the extracorporeal connector 20a and the hub 20b, as shown in FIG. 33A, or the hub 20b may incorporate the extracorporeal connector 20a. Fluid source 22 typically includes one or more sterile containers that hold the fluid(s) to be administered. Examples of typical sterile containers include plastic bags, glass bottles or plastic bottles.

An administration set 30 typically provides a sterile conduit for fluid to flow from fluid source 22 to cannula 20. Typically, administration set 30 includes tubing 32, a drip chamber 34, a flow control device 36, and a cannula connector 38. Tubing 32 typically is made of polypropylene, nylon, or another flexible, strong and inert material. Drip chamber 34 typically permits the fluid to flow one drop at a time for reducing air bubbles in the flow. Tubing 32 and drip chamber 34 typically are transparent or translucent to provide a visual indication of the flow. Typically, flow control device 36 controls fluid flow in tubing 32 and is positioned upstream from drip chamber 34. Roller clamps and Dial-A-Flo®, manufactured by Hospira, Inc. (Lake Forest, Ill., US), are examples of typical flow control devices. Typically, cannula connector 38 and extracorporeal connector 20a provide a leak-proof coupling through which the fluid may flow. Luer-Lok™, manufactured by Becton, Dickinson and Company (Franklin Lakes, N.J., US), is an example of a typical leak-proof coupling.

Administration set 30 may also include at least one of a clamp 40, an injection port 42, a filter 44, or other devices. Typically, clamp 40 pinches tubing 32 to cut-off fluid flow. Injection port 42 typically provides an access port for administering medicine or another fluid via cannula 20. Filter 44 typically purifies and/or treats the fluid flowing through administration set 30. For example, filter 44 may strain contaminants from the fluid.

An infusion pump 50 may be coupled with administration set 30 for controlling the quantity or the rate of fluid flow to cannula 20. The Alaris® System manufactured by CareFusion Corporation (San Diego, Calif., US), BodyGuard® Infusion Pumps manufactured by CMA America, L.L.C. (Golden, Colo., US), and Flo-Gard® Volumetric Infusion Pumps manufactured by Baxter International Inc. (Deerfield, Ill., US) are examples of typical infusion pumps.

Intravenous infusion or therapy typically uses a fluid (e.g., infusate, whole blood, or blood product) to correct an electrolyte imbalance, to deliver a medication, or to elevate a fluid level. Typical infusates predominately consist of sterile water with electrolytes (e.g., sodium, potassium, or chloride), calories (e.g., dextrose or total parenteral nutrition), or medications (e.g., anti-infectives, anticonvulsants, antihyperuricemic agents, cardiovascular agents, central nervous system agents, chemotherapy drugs, coagulation modifiers, gastrointestinal agents, or respiratory agents). Examples of medications that are typically administered during intravenous therapy include acyclovir, allopurinol, amikacin, aminophylline, amiodarone, amphotericin B, ampicillin, carboplatin, cefazolin, cefotaxime, cefuroxime, ciprofloxacin, cisplatin, clindamycin, cyclophosphamide, diazepam, docetaxel, dopamine, doxorubicin, doxycycline, erythromycin, etoposide, fentanyl, fluorouracil, furosemide, ganciclovir, gemcitabine, gentamicin, heparin, imipenem, irinotecan, lorazepam, magnesium sulfate, meropenem, methotrexate, methylprednisolone, midazolam, morphine, nafcillin, ondansetron, paclitaxel, pentamidine, phenobarbital, phenytoin, piperacillin, promethazine, sodium bicarbonate, ticarcillin, tobramycin, topotecan, vancomycin, vinblastine and vincristine. Transfusions and other processes for donating and receiving whole blood or blood products (e.g., albumin and immunoglobulin) also typically use intravenous infusion.

Unintended infusing typically occurs when fluid from cannula 20 escapes from its intended vein/artery. Typically, unintended infusing causes an abnormal amount of the fluid to diffuse or accumulate in perivascular tissue P and may occur, for example, when (i) cannula 20 causes a vein/artery to rupture; (ii) cannula 20 improperly punctures the vein/artery; (iii) cannula 20 backs out of the vein/artery; (iv) cannula 20 is improperly sized; (v) infusion pump 50 administers fluid at an excessive flow rate; or (vi) the infusate increases permeability of the vein/artery. As the terminology is used herein, “tissue” preferably refers to an association of cells, intercellular material and/or interstitial compartments, and “perivascular tissue” preferably refers to cells, intercellular material, interstitial fluid and/or interstitial compartments that are in the general vicinity of a blood vessel and may become unintentionally infused with fluid from cannula 20. Unintended infusing of a non-vesicant fluid is typically referred to as “infiltration,” whereas unintended infusing of a vesicant fluid is typically referred to as “extravasation.”

The symptoms of infiltration or extravasation typically include edema, pain or numbness in the vicinity of the cannulation site N; blanching, discoloration, inflammation or coolness of the skin S in the vicinity of the cannulation site N; breakdown, tautness or stretching of the skin S; or drainage from the cannulation site N. The consequences of infiltration or extravasation typically include skin reactions (e.g., blisters), nerve compression, compartment syndrome, or necrosis. Typical treatments for infiltration or extravasation include (i) applying warm or cold compresses; (ii) elevating the affected limb; (iii) administering hyaluronidase, phentolamine, sodium thiosulfate or dexrazoxane; (iv) fasciotomy; or (v) amputation.

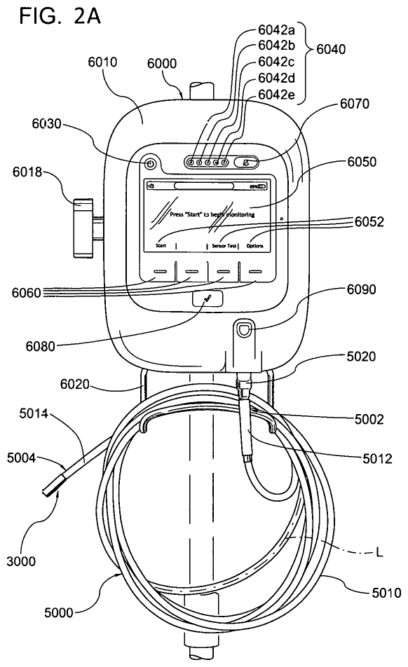

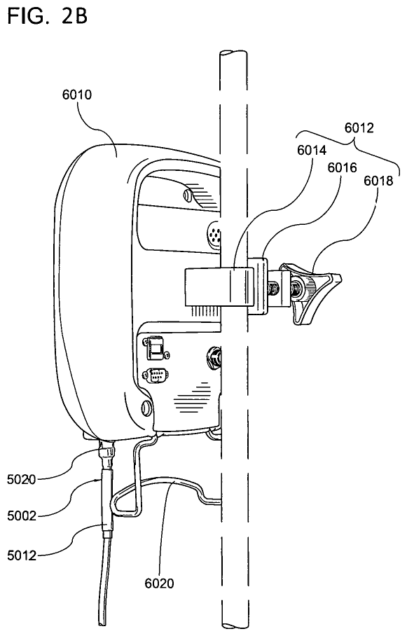

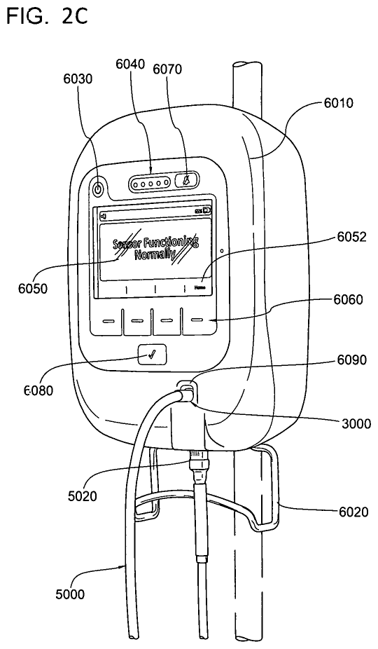

FIGS. 2A-2C show a known patient monitoring device 6000 manufactured by ivWatch, LLC (Hampton, Va., USA) to preferably aid in diagnosing at least one of infiltration and extravasation in Animalia tissue. Patient monitoring device 6000 preferably includes a number of features disposed on an exterior of shell 6010. Preferably, patient monitoring device 6000 includes a power button 6030, an indicator set 6040, a display 6050, a set of soft keys 6060, a mute button 6070, a check button 6080 and a test port 6090. According to the embodiment shown in FIG. 2A, these features preferably are disposed on the front of shell 6010. Pressing power button 6030 preferably toggles ON and OFF patient monitoring device 6000.

Patient monitoring device 6000 preferably provides status reports of varying detail. Preferably, indicator set 6040 provides a basic status report and display 6050 provides a more detailed status report. According to one embodiment, indicator set 6040 includes a set of multi-color light emitting diodes 6042a-6042e providing a visible indication of at least one of three states of patient monitoring device 6000. A first state of patient monitoring device 6000 preferably includes all of multi-color light emitting diodes 6042a-6042e illuminating a first color, e.g., green. Preferably, the first state is characterized by actively monitoring for indications of infiltration or extravasation without identifying a cause for alerting a healthcare giver to evaluate the patient. A second state of patient monitoring device 6000 preferably includes all of multi-color light emitting diodes 6042a-6042e illuminating a second color, e.g., yellow. Preferably, the second state is characterized by identifying a cause for alerting the healthcare giver to evaluate the operation of system 100 with respect to the patient. For example, the second state may be indicated if the operation of system 100 is being disrupted because the patient is pulling on sensor cable 5000. A third state of patient monitoring device 6000 preferably includes all of multi-color light emitting diodes 6042a-6042e illuminating a third color, e.g., red. Preferably, the third state is characterized by patient monitoring device 6000 alerting the healthcare giver to perform an infiltration/extravasation examination. According to other embodiments, the number as well as color(s) of multi-color light emitting diodes 6042a-6042e that are illuminated may provide information regarding, for example, duration or intensity of an event that is cause for alerting a healthcare giver.

Display 6050 preferably provides detailed information regarding the use, status, and alarms of patient monitoring device 6000. Preferably, display 6050 includes color, alphanumeric characters, graphs, icons and images to convey set-up and operating instructions, system maintenance and malfunction notices, system configuration statements, healthcare giver alerts, historical records, etc. According to one embodiment, display 6050 preferably displays individual labels 6052 describing a function assigned to a corresponding soft key 6060. According to other embodiments, display 6050 preferably facilitates quantifying with precision when an identifiable event occurred, its duration, its magnitude, whether an alert was issued, and the corresponding type of alert.

Mute button 6070 and check button 6080 preferably are hard keys having regularly assigned functions. Preferably, mute button 6070 temporarily silences an audible alarm. According to one embodiment, a healthcare giver preferably silences the audible alarm while performing an infiltration/extravasation examination. Preferably, the function of mute button 6070 is temporary because disabling rather than silencing the audible alarm may be detrimental to the future effectiveness of patient monitoring device 6000. Check button 6080 preferably includes one or more regularly assigned functions, e.g., registering periodic examinations of the cannulation site N. According to one embodiment, check button 6080 is preferably pressed each time a healthcare giver performs an examination of the cannulation site N. Preferably, the examination is registered in a historical record maintained by patient monitoring device 6000. According to other embodiments, the historical record may be reviewed on display 6050 and/or the historical record may be transferred off patient monitoring device 6000 to a recordkeeping system that maintains a generally comprehensive chronicle of the patient's treatment(s).

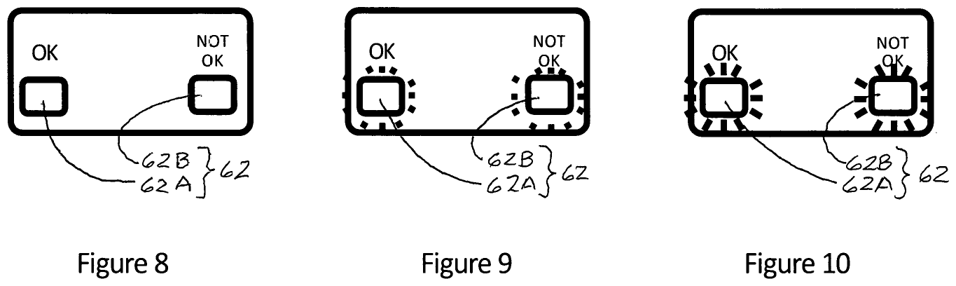

The features described and shown in FIGS. 2A-2C illustrate an example of a physical environment in which the present invention may be used, but do not describe or show the inventive features according to the present disclosure. The inventive features of the present disclosure will be described with regard to a first embodiment shown in FIGS. 3-7 and a second embodiment shown in FIGS. 8-13.

A system of annotating a patient medical record according to a first embodiment will now be described with reference to the schematic illustration of a user interface shown in FIGS. 3-7. A display 50 preferably displays individual labels 52 describing a function assigned to a corresponding soft key 60. Although four pairs of corresponding labels 52 and keys 60 are shown, fewer or more pairs are envisioned according to the present invention. A check button 80 preferably has a surround ring 82 that can be illuminated. Alternatively, an illuminating device, an audible device, or some other form of indicator may be substituted for ring 82.

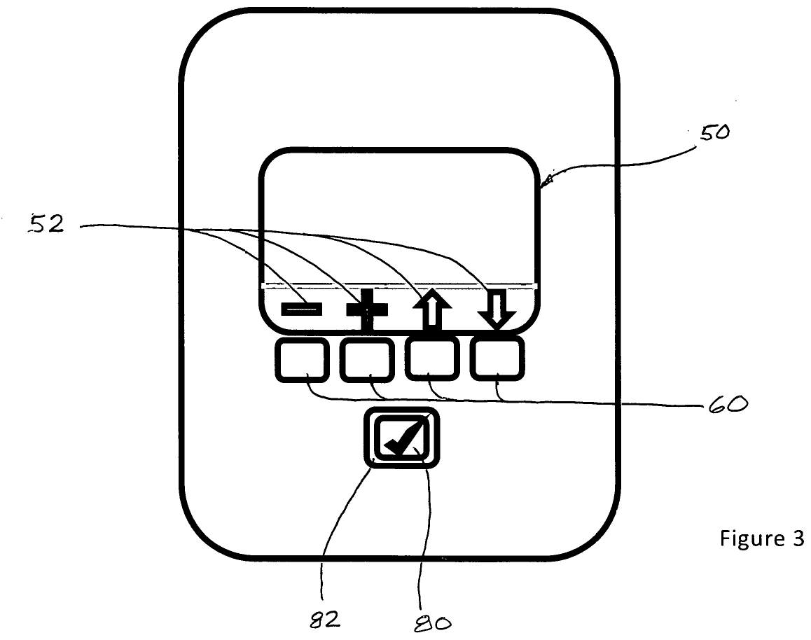

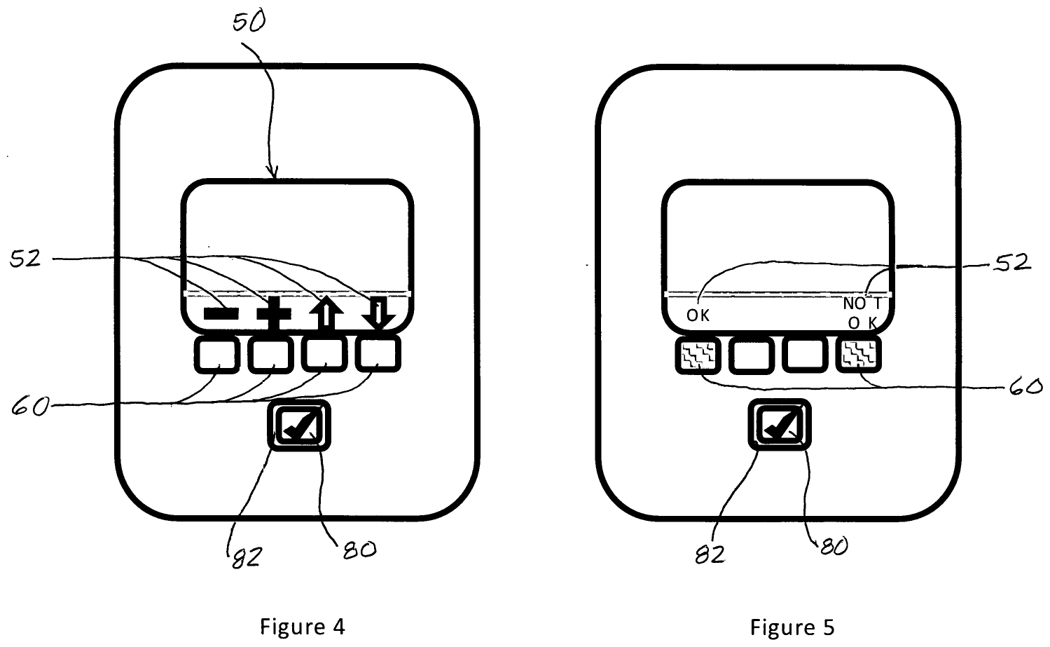

FIG. 3 shows a nominal state of the user interface. FIG. 4 shows button 80 in a pressed state—this is illustrated with grey—then the labels 52 change to OK and NOT OK as shown in FIG. 5. There are two keys 60 illustrated with grey stripes—this illustrates the option to press one or the other—are also shown in FIG. 5. One grey stripped key 60 is below the individual OK and NOT OK labels 52 shown on the display 50.

Based on the caregiver's evaluation of the patient's intravascular infusion, the caregiver selects either the OK key 60 or the NOT OK key 60. The act or pressing button 80 preferably generates a date/time stamp. The act of pressing the OK key 60 preferably generates an acknowledgement of a healthy infusion, or the act of pressing the NOT OK preferably generates a place-holder for additional information to be generated. Because of the general nature of OK, the patient's medical record may automatically be annotated with standard language of the health care provider or caregiver. Thus, pressing the OK key 60 may cause the patient's medical record, whether on the local device and/or remotely located storage source, to be annotated with the date/time stamp and a phrase such as, for example, “Patient appears to be progressing as anticipated.” The diverse nature of pressing the NOT OK key 60 preferably annotates the patient's medical record with the date/time stamp and a prompt for the caregiver to complete the annotation. The prompt may be maintained internally in the patient's medical record and/or disseminated externally, e.g., via email or text, to facilitate reminders for the caregiver to complete the annotation.

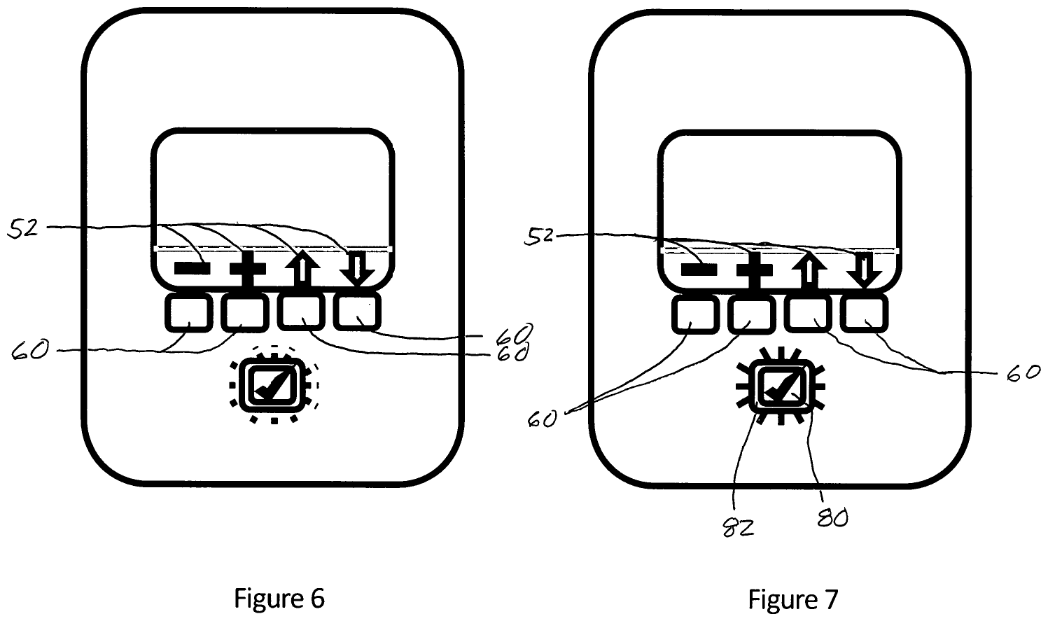

FIG. 6 shows a variation where the ring 82 flashes after a certain time period has passed. Flashing of the ring 82—shown with broken radiating lines—preferably indicates to the caregiver that at least a minimum amount of time has passed since the last evaluation of the patient's intravascular infusion. Pressing button 80 discontinues ring 82 flashing and presents the state shown in FIG. 5, i.e., the device is ready to accept the caregiver's selection of either OK key 60 or NOT OK key 60.

FIG. 7 shows a further variation where the ring 82 is steadily illuminated after a certain time period has passed. Steady illumination of the ring 82—shown with solid radiating lines—preferably indicates to the caregiver that at least a maximum amount of time has passed since the last evaluation of the patient's intravascular infusion, i.e., re-evaluation is overdue. Pressing button 80 discontinues ring 82 being illuminated and presents the state shown in FIG. 5, i.e., the device is ready to accept the caregiver's selection of either OK key 60 or NOT OK key 60.

A system of annotating a record according to a second embodiment will now be described with reference to the schematic illustration of a user interface shown in FIGS. 8-13. Only two pushbuttons 62 individually labelled OK (pushbutton 62A) and NOT OK (pushbutton 62B) are included in the user interface according to the present invention. Preferably, each of the pushbuttons 62 includes face with an illumination feature that can flash or steadily illuminate the face.

FIG. 8 shows a nominal state of the user interface. Pressing the OK pushbutton 62A in the nominal state will annotate the associated record with a date/time stamp and a phrase such as, for example, “Acceptable evaluation.” Pressing the NOT OK pushbutton 62B in the nominal state will annotate the associated record with a date/time stamp and a prompt to complete the annotation.

FIG. 9 shows both pushbuttons 62 flashing to indicate a certain time period has passed since the last evaluation was noted. Preferably, flashing indicates that at least a minimum amount of time has passed since the last evaluation occurred. Pressing the OK pushbutton 62A in the flashing state will annotate the associated record with a date/time stamp and a phrase such as, for example, “Acceptable evaluation.” Pressing the NOT OK pushbutton 62B in the flashing state will annotate the associated record with a date/time stamp and a prompt to complete the annotation. Pressing either pushbutton 62 discontinues the flashing.

FIG. 10 shows both pushbuttons 62 steadily illuminated to indicate a certain time period has passed since the last evaluation was noted. Preferably, steady illumination indicates that at least a maximum amount of time has passed since the last evaluation, i.e., re-evaluation is overdue. Pressing the OK pushbutton 62A in the steadily illuminated state will annotate the associated record with a date/time stamp and a phrase such as, for example, “Acceptable evaluation.” Pressing the NOT OK pushbutton 62B in the steadily illuminated state will annotate the associated record with a date/time stamp and a prompt to complete the annotation. Pressing either pushbutton 62 discontinues the steadily illumination.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An annotation system for an electronic medical record of a patient, the system comprising:
a patient monitoring device aiding detection of at least one of infiltration and extravasation at a cannulation site of the patient; and
a user interface controlling the patient monitoring device, the user interface including—
a check button having at least one regularly assigned function including registering periodic examinations of the cannulation site;
a first key annotating the electronic medical record with an indication that the cannulation site is healthy; and
a second key annotating the electronic medical record with an indication that at least one of infiltration and extravasation is indicated at the cannulation site.

2. The system of claim 1 wherein the first key annotates the electronic medical record with a predetermined annotation.

3. The system of claim 1 wherein the second key annotates the electronic medical record with a prompt to complete an annotation.

4. The system of claim 1 wherein the first and second keys annotates the electronic medical record with a date/time stamp.
